(19) United States
(12) Patent Application Publication
(10) Pub. No.: US 2024/0209423 A1
(43) Pub. Date: Jun. 27, 2024

(54) METHODS AND COMPOSITIONS FOR REDUCING AUTOFLUORESCENCE

(71) Applicant: Advanced Cell Diagnostics, Inc., Newark, CA (US)

(72) Inventors: Li-chong Wang, Newark, CA (US); Han Lu, Newark, CA (US); Xiao-Jun Ma, Newark, CA (US); Bing-Qing Zhang, Newark, CA (US); Hailing Zong, Newark, CA (US); Ha Yeun Ji, Newark, CA (US); Ming Yu, Newark, CA (US)

(21) Appl. No.: 18/555,657

(22) PCT Filed: Apr. 15, 2022
(86) PCT No.: PCT/US22/24978

§ 371 Date: Oct. 16, 2023

Related U.S. Application Data
(60) Provisional application No. 63/175,656, filed on Apr. 16, 2021.

(51) Int. Cl. C12Q 1/6832

(52) U.S. Cl. CPC C12Q 1/6832

(57) ABSTRACT

The present disclosure provides methods and compositions for the reduction of autofluorescence in biological samples, such as tissue samples.

### CROSS-REFERENCE TO RELATED APPLICATIONS

This application claims priority to and the benefit of U.S. Provisional Patent Application No. 63/175,656, filed on Apr. 16, 2021, which is incorporated herein by reference in its entirety.

### FIELD

The present disclosure provides methods and compositions for the reduction of autofluorescence in biological samples, such as tissue samples.

### BACKGROUND

Background fluorescence, such as tissue autofluorescence, is a long-standing challenge in the field of fluorescent-based imaging of cells and tissue samples, including methods of detecting proteins (e.g., immunofluorescence (IF)), nucleic acids (e.g., fluorescence in situ hybridization (FISH)), and co-detection methods. Such assays utilize fluorescent dyes and fluorescent microscopes for target labeling and detection in the tissue context, respectively. Due to the inherent nature of autofluorescence within tissue samples, it interferes with detection of true signals in IF and FISH assays, especially when the signal of interests are low (e.g., less abundant). Tissue autofluorescence is widely observed in pathology samples prepared by widely used methods, such as in formalin-fixed paraffin-embedded (FFPE) tissue specimens, which is particularly prone to high tissue autofluorescence due to long fixation and processing steps involved. In addition, autofluorescence is present in some fixed frozen and fresh frozen tissues.

For example, tissue autofluorescence has been attributed to natural fluorescence that come from endogenous components in cells such as aromatic amino acids, lipopigments, the extracellular matrix components, as well as fluorescence generated during fixation procedures. In FFPE tissues, autofluorescence is observed from lysosomal digestion residues called lipofuscins, the ECM protein collagens and elastins, red blood cells, as well as from formalin fixation. Autofluorescence has a broad excitation and emission spectrum around the FITC and Cy3 channels, and thus hampers the clear visualization of fluorescent RNA and protein signals in these spectra.

Several methods have been developed to mitigate tissue autofluorescence. For example, the difference between signal intensity and autofluorescence background can be experimentally increased by boosting signal intensity (e.g., through the use of tyramide signal amplification), and/or by reducing autofluorescence background while preserving signal. Current commercially available products that employ an autofluorescence reduction strategy include Sudan Black, TrueBlack®, and TrueVIEW®. While they all reduce autofluorescence to certain extent, there are significant limitations with these products. For example, Sudan Black shows undesired shifting of spectrum into the far-red channel, TrueBlackt specifically quenches lipofuscin autofluorescence but is not effective on autofluorescence produced from fixative background, and TrueVIEW® efficiently quenches all sources of autofluorescence, but significantly dampens target signal intensity. Furthermore, these treatments have significant variation across different tissues, making it difficult to apply a universal and robust autofluorescence blocking procedure. Additionally, post-image processing can be performed to reduce autofluorescence using various methods, including spectral imaging and unmixing or autofluorescence removal software, (e.g., dotdotdot and AFid). However, these methods either require special, high-end microscopic setups or are only effective in removing highly autofluorescent sources such as red blood cells, but not in removing fixative-induced general background. Hence, there is a critical need to develop methods that mitigate the impact of tissue autofluorescence while maintaining the fluorescent signals from desired targets.

### SUMMARY

Embodiments of the present disclosure include compositions and methods for reducing autofluorescence in biological samples.

In one aspect, the disclosure provides a method of detecting a target in a biological sample, the method comprising: contacting the sample with a probe that binds to the target; contacting the sample with a quinone compound; and detecting a signal corresponding to the target in the sample.

In some embodiments, the target is a nucleic acid target. In some embodiments, the probe is a first target probe comprising a T section and an L section, wherein the T section is complementary to a portion of the nucleic acid target and wherein the L section is complementary to a nucleic acid component of a signal generating complex. In some embodiments, the method further comprises contacting the sample with a second target probe comprising a T section and an L section, wherein the T section is complementary to a portion of the nucleic acid target and wherein the L section is complementary to a nucleic acid component of a signal generating complex. In some embodiments, the T sections of the first target probe and the second target probe are complementary to non-overlapping regions of the nucleic acid target. In some embodiments, the L sections of the first target probe and the second target probe are complementary to non-overlapping regions of the signal generating complex.

In some embodiments, the method further comprises contacting the sample with a signal generating complex comprising at least one detectable label. In some embodiments, the detecting step comprises detecting a signal generated by the signal generating complex. In some embodiments, the signal generating complex comprises a label probe that binds to the detectable label, and optionally, one or more of an amplifier, a pre-amplifier, and a pre-preamplifier. In some embodiments, the signal generating complex comprises a label probe, an amplifier, and a pre-amplifier. In some embodiments, the sample is contacted with the quinone compound after the sample has been contacted with the first target probe and the second target probe, but before the sample is contacted with the detectable label. In some embodiments, the detectable label is a fluorophore or a quantum dot. In some embodiments, the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

In some embodiments, the nucleic acid target is selected from an RNA and a DNA. In some embodiments, the nucleic acid target is an RNA, and the RNA is selected from messenger RNA (mRNA), microRNA (miRNA), ribosomal RNA (rRNA), mitochondrial RNA, and non-coding RNA. In some embodiments, the nucleic acid target is mRNA. In some embodiments, the nucleic acid target is DNA.

In some embodiments, the target is a protein. In some embodiments, the probe is a primary antibody that binds to the protein target, and the method further comprises contacting the sample with a secondary antibody that binds to the primary antibody, wherein the secondary antibody binds to a detectable label.

In some embodiments, the method comprises detecting multiple targets in the biological sample. In some embodiments, the method comprises detecting multiple nucleic acid targets, multiple protein targets, or at least one each of a nucleic acid target and a protein target.

In some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone.

In some embodiments, the sample is a tissue specimen or is derived from a tissue specimen. In some embodiments, the sample is a formalin fixed paraffin embedded tissue specimen, a fixed frozen tissue specimen, or a fresh frozen tissue specimen. In some embodiments, sample is a formalin fixed paraffin embedded tissue specimen, and the method further comprises performing a deparaffinization step prior to the contacting or detecting steps.

In one aspect, the disclosure provides a kit for detecting a target in a biological sample, the kit comprising: a probe that binds to the target; a quinone compound; and instructions for conducting an assay to detect the target in the biological sample.

In some embodiments, the target is a nucleic acid target. In some embodiments, the probe is a first target probe comprising a T section and an L section, wherein the T section is complementary to a portion of the nucleic acid target and wherein the L section is complementary to a nucleic acid component of a signal generating complex. In some embodiments, the kit further comprises a second target probe comprising a T section and an L section, wherein the T section is complementary to a portion of the nucleic acid target and wherein the L section is complementary to a nucleic acid component of a signal generating complex. In some embodiments, the T sections of the first target probe and the second target probe are complementary to non-overlapping regions of the nucleic acid target. In some embodiments, the L sections of the first target probe and the second target probe are complementary to non-overlapping regions of the signal generating complex.

In some embodiments, the kit further comprises a signal generating complex comprising at least one detectable label. In some embodiments, the signal generating complex comprises a label probe that binds to the detectable label, and optionally, one or more of an amplifier, a pre-amplifier, and a pre-pre-amplifier. In some embodiments, the signal generating complex comprises a label probe, an amplifier, and a pre-amplifier. In some embodiments, the detectable label is a fluorophore or a quantum dot. In some embodiments, the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

In some embodiments, the nucleic acid target is selected from an RNA and a DNA. In some embodiments, the nucleic acid target is an RNA, and the RNA is selected from messenger RNA (mRNA), microRNA (miRNA), ribosomal RNA (rRNA), mitochondrial RNA, and non-coding RNA. In some embodiments, the nucleic acid target is mRNA. In some embodiments, the nucleic acid target is DNA.

In some embodiments, the target is a protein. In some embodiments, the probe is a primary antibody that binds to the protein target, and the kit further comprises a secondary antibody that binds to the primary antibody, wherein the secondary antibody binds to a detectable label.

In some embodiments, the kit further comprises a second probe for a second target in the biological sample. In some embodiments, the second target is a nucleic acid or a protein. In some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone. In some embodiments, the kit comprises a composition of the quinone compound at a concentration of about 1 mM to about 100 mM, or about 10 mM to about 150 mM, e.g., about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM.

In some embodiments, the kit further comprises at least one of a hybridization buffer, dextran sulfate, formamide, dithiothreitol (DDT), sodium chloride and sodium citrate (SSC), EDTA, Denhardt's solution, dNTPs, single-stranded DNA, tRNA, polyA, an initiator oligo, or any combination thereof.

In one aspect, the disclosure provides composition comprising: a biological sample comprising a target; a probe that binds to the target; and a quinone compound.

In some embodiments, the target is a nucleic acid target. In some embodiments, the probe is a first target probe comprising a T section and an L section, wherein the T section is complementary to a portion of the nucleic acid target and wherein the L section is complementary to a nucleic acid component of a signal generating complex. In some embodiments, further comprising a second target probe comprising a T section and an L section, wherein the T section is complementary to a portion of the nucleic acid target and wherein the L section is complementary to a nucleic acid component of a signal generating complex. In some embodiments, the T sections of the first target probe and the second target probe are complementary to non-overlapping regions of the nucleic acid target. In some embodiments, the L sections of the first target probe and the second target probe are complementary to non-overlapping regions of the signal generating complex.

In some embodiments, the composition further comprises a signal generating complex comprising at least one detectable label. In some embodiments, the signal generating complex comprises a label probe that binds to the detectable label, and optionally, one or more of an amplifier, a pre-amplifier, and a pre-pre-amplifier. In some embodiments, the signal generating complex comprises a label probe, an amplifier, and a pre-amplifier. In some embodiments, the detectable label is a fluorophore or a quantum dot. In some embodiments, the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

In some embodiments, the nucleic acid target is selected from an RNA and a DNA. In some embodiments, the nucleic acid target is an RNA, and the RNA is selected from messenger RNA (mRNA), microRNA (miRNA), ribosomal RNA (rRNA), mitochondrial RNA, and non-coding RNA. In some embodiments, the nucleic acid target is mRNA. In some embodiments, the nucleic acid target is DNA.

In some embodiments, the target is a protein. In some embodiments, the probe is a primary antibody that binds to the protein target, and the composition further comprises a secondary antibody that binds to the primary antibody, wherein the secondary antibody binds to a detectable label.

In some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone.

In some embodiments, the biological sample is a tissue specimen or is derived from a tissue specimen. In some embodiments, the sample is a formalin fixed paraffin embedded tissue specimen, a fixed frozen tissue specimen, or a fresh frozen tissue specimen. In some embodiments, the biological sample is a cytological sample or is derived from a cytological sample.

In another aspect, the disclosure provides a method of reducing autofluorescence in a biological sample, comprising contacting the sample with an effective amount of a quinone compound. In some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone. In some embodiments, the sample is a tissue specimen or is derived from a tissue specimen. In some embodiments, the sample is a formalin fixed paraffin embedded tissue specimen, a fixed frozen tissue specimen, or a fresh frozen tissue specimen.

Other aspects and embodiments of the disclosure will be apparent in light of the following detailed description.

### BRIEF DESCRIPTION OF THE DRAWINGS

FIG. 1 shows a schematic of an exemplary RNA probe hybridization and amplification system.

FIG. 2 shows a schematic of a workflow for quinone compound application in a RNA detection assay, where the quinone compound application reduces tissue autofluorescence.

FIG. 3 shows images demonstrating that p-benzoquinone treatment reduced autofluorescence in mouse formalin fixed paraffin embedded (FFPE) kidney tissue in an RNA in situ hybridization assay.

FIG. 4 shows images demonstrating that p-benzoquinone treatment reduced autofluorescence in human FFPE lung cancer tissue in an RNA in situ hybridization assay.

FIG. 5 shows images demonstrating that p-benzoquinone treatment reduced autofluorescence in non-human primate fixed frozen tissue in an RNA in situ hybridization assay.

FIG. 6 shows images demonstrating that p-benzoquinone treatment is compatible with an RNA in situ hybridization assay in mouse fresh frozen tissue.

FIGS. 7A-7D show images demonstrating that p-benzoquinone is compatible with certain fluorescent dyes.

FIG. 8 shows images demonstrating that p-benzoquinone autofluorescence quenching treatment is compatible with immunofluorescence assays.

FIGS. 9A-9E show images demonstrating that p-benzoquinone partially quenches lipofuscin autofluorescence.

FIGS. 10A-10B show representative workflow diagrams of the image processing methods to reduce background signals, described with reference to the images being processed (FIG. 10A) and the general steps of the method (FIG. 10B), according to one embodiment of the present disclosure.

### DETAILED DESCRIPTION

The present disclosure provides compositions and methods related to the reduction of autofluorescence in samples, such as biological samples, which can allow for enhanced signal detection in both in situ hybridization (ISH) and immunocytochemical (ICC)/immunofluorescent (IF) assays. In particular, the present disclosure provides compositions and methods for detecting targets in biological samples, in which the sample is contacted with a quinone compound, such as hydroquinone or p-benzoquinone, to reduce autofluorescence in the sample. Use of the quinone compound can rapidly and effectively reduce autofluorescence while maintaining signals from targets in the biological samples, such as signals resulting from the detection of nucleic acids and proteins.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the present disclosure may be readily combined, without departing from the scope or spirit of the embodiments provided herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has.” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “in situ hybridization” or “ISH” refers to a technique for localizing and visualizing specific target nucleic acids with the preservation of morphology of the source samples. In some embodiments, “in situ hybridization” comprises fluorescent detection, which is generally referred to as “fluorescent in situ hybridization” (FISH).

Immunocytochemistry (ICC), Immunohistochemistry (IHC) and Immunofluorescence (IF) all utilize antibodies to provide details and characteristics about peptides, polypeptides, and proteins, including but not limited to, abundance, distribution, and localization. As used herein, the term “immunohistochemistry” or “IHC” generally refers to a technique for detecting peptides, polypeptide, and proteins of interest in source samples utilizing antibodies, with the preservation of morphology of the source samples, which is generally tissue samples. As used herein, the term “immunocytochemistry” or “ICC” generally refers to the staining of isolated or cultured intact cells where samples may be from tissue culture cell lines, either adherent or in suspension. Immunofluorescence (IF) refers to fluorescent labeling, thus it is also encompassed in IHC and ICC processes/assays. ICC, IHC, and IF assays can be used in conjunction with the imaging processing methods of the present disclosure, as described further herein, including facilitating quantitative and/or qualitative assessments of a target-of-interest in a sample. ICC, IHC, and IF assays can also be performed in conjunction with an in situ hybridization as part of an integrated co-detection process to detect targets-of-interest, which can also include performing the imaging processing methods of the present disclosure.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “probe” as used herein refers to a capture agent that is directed to a specific target in a biological sample, such as the nucleic acid target or a target protein. In some embodiments, the probe provided herein is a “nucleic acid probe” or “oligonucleotide probe” which refers to a nucleic acid capable of binding to a target nucleic acid of complementary sequence, such as a mRNA sequence or a DNA sequence, usually through complementary base pairing by forming hydrogen bond. As used herein, a nucleic acid probe may include natural (e.g., A, G, C, or T) or modified bases (7-deazaguanosine, inosine, etc.). In addition, the bases in a nucleic acid probe may be joined by a linkage other than a phosphodiester bond, so long as it does not interfere with hybridization. In some embodiments, the probe provided herein is an antibody, which can bind to a target protein. The probes can be directly or indirectly labeled with tags, for example, chromophores, lumiphores, or chromogens. By assaying for the presence or absence of the probe, one can detect the presence or absence of a target of interest.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A “single-stranded nucleic acid” having secondary structure (e.g., base-paired secondary structure) and/or higher order structure comprises a “double-stranded nucleic acid.” For example, triplex structures are considered to be “double-stranded.” In some embodiments, any base-paired nucleic acid is a “double-stranded nucleic acid.”

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

In some embodiments the substitutions can be conservative amino acid substitutions. Examples of conservative amino acid substitutions, unlikely to affect biological activity, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and these changes in the reverse. See e.g., Neurath et al., The Proteins, Academic Press, New York (1979), the relevant portions of which are incorporated herein by reference. Further, an exchange of one amino acid within a group for another amino acid within the same group is a conservative substitution, where the groups are the following: (1) alanine, valine, leucine, isoleucine, methionine, norleucine, and phenylalanine; (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary,” “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that arc complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like.

As used herein, the term “primary antibody” generally refers to an antibody that binds directly to an antigen of interest. As used herein, the term “secondary antibody” refers to an antibody that is conjugated to a detectable label. In some embodiments, the secondary antibody provided herein binds directly to the primary antibody. In other embodiments, the secondary antibody provided herein binds indirectly to the primary antibody (e.g., by binding to another antibody that recognizes the primary antibody).

The term “detecting” as used herein generally refers to any form of measurement, and include determining whether an element is present or not. This term includes quantitative and/or qualitative determinations.

The term “quinone compound” as used herein refers to a compound having a fully conjugated cyclic dione structure, derived from a compound comprising an aromatic ring in which an even number of —CH═ groups are converted to —C(═O)— groups, with any necessary rearrangement of double bonds. An exemplary quinone compound is p-benzoquinone; others are described herein, including polycyclic and substituted analogs. As used herein, the term “quinone compound” also encompasses the reduced forms of such compounds (i.e., the corresponding diol), such as hydroquinone.

The term “network” as used herein generally refers to any suitable electronic network including, but not limited to, a wide area network (“WAN”) (e.g., a TCP/IP based network), a local area network (“LAN”), a neighborhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc.

The term “computer” as used herein generally includes a plurality of electrical and electronic components that provide power, operational control, and protection to the components and modules within the system. For example, a computer can include, among other things, a processing unit (e.g., a microprocessor, a microcontroller, or other suitable programmable device), a memory, input units, and output units. The processing unit can include, among other things, a control unit, an arithmetic logic unit (“ALC”), and a plurality of registers, and can be implemented using a known computer architecture (e.g., a modified Harvard architecture, a von Neumann architecture, etc.).

The term “memory” as used herein generally refers to any memory storage of the computer and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processing unit can be connected to the memory and execute software instructions that are capable of being stored in a RAM of the memory (e.g., during execution), a ROM of the memory (e.g., on a generally permanent bases), or another non-transitory computer readable medium such as another memory or a disc. Software included in the implementation of the methods disclosed herein can be stored in the memory. The software includes, for example, firmware, one or more applications, program data, filters, rules, one or more program modules, and other executable instructions. For example, the computer can be configured to retrieve from the memory and execute, among other things, instructions related to the processes and methods described herein.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. COMPOSITIONS

Embodiments of the present disclosure provide compositions and methods related to detecting one or more targets in a sample, in which autofluorescence in the sample is reduced. In some embodiments, of the compositions provided herein enable the detection of a target in a biological sample, such as a nucleic acid target or a protein target.

In accordance with these embodiments, the present disclosure includes compositions that include a biological sample comprising a target, a probe that binds to the target, and a quinone compound. The inclusion of the quinone compound reduces autofluorescence in the biological sample, allowing for improved detection of signals corresponding to the target in the sample.

In some embodiments, the target in the biological sample is a nucleic acid, which can be detected using a technique such as in situ hybridization (a method which is described further herein below). In particular embodiments, the probe in the composition comprises a target (T) section and a label (L) section. The T section is complementary to a portion of a first domain of a target nucleic acid (e.g., a target mRNA) and the L section is complementary to a nucleic acid component of a signal generating complex (SGC). In some embodiments, the composition further comprises a second target probe comprising a T section and an L section, wherein the T section is complementary to a portion of the nucleic acid target and wherein the L section is complementary to a nucleic acid component of a signal generating complex. In some embodiments, the T sections of the first target probe and the second target probe are complementary to non-overlapping regions of the target nucleic acid, and the L sections of the first target probe and the second target probe are complementary to non-overlapping regions of the nucleic acid component of the signal generating complex.

In particular, the hybridization or binding of the first and second probes to their respective T sections in a target nucleic acid facilitates the generation of two adjacent L sections to which a complementary nucleic acid component of a signal generating complex (SGC) can bind. The SGC includes at least one detectable label. In some embodiments, the nucleic acid portion of the signal generating complex binds both the L sections of the first target probe and the second target probe. In some embodiments, a first nucleic acid component of the signal generating complex binds to the L section of the first target probe, and a second nucleic acid component of the signal generating complex binds to the L section of the second target probe.

In some embodiments, once a component of the SGC binds the target probes at their L sections, other components of the SGC can bind. In some embodiments, the SGC provided herein comprises additional components, including but not limited to an amplifier, a pre-amplifier, and/or a pre-pre-amplifier. In accordance with these embodiments, the formation of the SGC allows for the generation of a detectable signal that is indicative of the presence of the target nucleic acid. Detecting the nucleic acid using a SGC and/or a label probe an occur by means well known in the art, including but not limited to, chromogenic or fluorescent detection means (described further below).

In some embodiments, the T section of the first target probe is 3′ of its L section. In some embodiments, the T section of the first target probe is 5′ of its L section. In some embodiments, the T section of the second target probe is 3′ of its L section. In some embodiments, the T section of the second target probe is 5′ of its L section. In some embodiments, the T section of first target probe is 3′ of its L section, and the T section of the second target probe is 3′ of its L section, or vice versa (e.g., both probes have the same directionality). In some embodiments, the T section of the first target probe is 5′ of its L section, and the T section of the second target probe is 5′ of its L section, or vice versa (e.g., both probes have the same directionality). In some embodiments, the T section of the first target probe is 5′ of its L section, and the T section of the second target probe is 3′ of its L section, or vice versa (e.g., one probe has directionality that is opposite the other). In some embodiments, the T section of the first target probe is 3′ of its L section, and the T section of the second target probe is 5 of its L section, or vice versa (e.g., one probe has directionality that is opposite the other).

In some embodiments, first target probe and/or the second target probe form a hairpin structure (e.g., useful for performing an assay involving hybridization chain reaction). In some embodiments, first target probe and/or the second target probe comprises a non-binding portion separating its T section from its L section (e.g., a spacer or linker region). In some embodiments, this non-binding portion does not bind to either the target nucleic acid or the nucleic acid component of the SGC, but is useful in forming the SGC. In some embodiments, this non-binding region can form the unpaired loop section of a hairpin loop. In other embodiments, neither the target probes contains a non-binding portion.

In some embodiments, the SGC includes a label probe that binds to the detectable label (e.g., has a detectable label covalently attached to the label probe, or can non-covalently bind to a detectable label), and optionally, one or more of an amplifier, a pre-amplifier, and a pre-pre-amplifier. In some embodiments, the SGC comprises a label probe, an amplifier, and a pre-amplifier. In some embodiments, the SGC comprises a label probe, an amplifier, a pre-amplifier, and a pre-pre-amplifier. In some embodiments, the SGC does not bind stably to either the L section of the first target probe alone or the L section of the second target probe alone. In such embodiments, although the SGC may transiently bind to the L section of either the first target probe or the second target probe alone, it will not remain bound; the interaction is only stable when both target probes are present. In some embodiments, the label probe includes at least one detectable label (e.g., chromogenic or fluorescent detection means).

In some embodiments, the composition further includes one or more components useful for carrying out a nucleic acid hybridization reaction, such as an in situ hybridization reaction or a hybridization chain reaction assay. The composition can include, but is not limited to, one or more of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In some embodiments, the nucleic acid target is an RNA, and the RNA is selected from messenger RNA (mRNA), microRNA (miRNA), ribosomal RNA (rRNA), mitochondrial RNA, and non-coding RNA (e.g., long non-coding RNA (lncRNA)). In some embodiments, the nucleic acid target is mRNA. In some embodiments, the nucleic acid target is DNA.

In accordance with these embodiments, when the target is a nucleic acid target, the “target probes” contained in the compositions of the present disclosure generally refer to a polynucleotide that is capable of hybridizing to a target nucleic acid and capturing or binding a label probe or SGC component to that target nucleic acid. The target probe can be bound directly to a detectable label, or it can hybridize directly to the label probe, or it can hybridize to one or more nucleic acids that in turn hybridize to the label probe; for example, the target probe can hybridize to an amplifier, a pre-amplifier or a pre-pre-amplifier in a SGC. Thus, in some embodiments, the target probe includes a first polynucleotide sequence that is complementary to a polynucleotide sequence of the target nucleic acid (T section), and a detectable label. In some embodiments, the target probe includes a first polynucleotide sequence that is complementary to a polynucleotide sequence of the target nucleic acid (T section) and a second polynucleotide sequence that is complementary to a polynucleotide sequence of the label probe (L section), amplifier, pre-amplifier, pre-pre-amplifier, or the like. The target probe is generally single-stranded so that the complementary sequence is available to hybridize with a corresponding target nucleic acid, label probe, amplifier, pre-amplifier or pre-pre-amplifier. In some embodiments, the target probes are provided as a pair.

In some embodiments, the compositions of the present disclosure include a “label probe.” which generally refers to a polynucleotide that binds to a target molecule, directly or indirectly, generally indirectly, and allows the target to be detected. A label probe contains a nucleic acid binding portion that is typically a single stranded polynucleotide or oligonucleotide that comprises one or more labels which directly or indirectly provide a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. In some embodiments, the label probe can comprise at least one polynucleotide sequence that is complementary to a polynucleotide sequence in an amplifier, pre-amplifier, or pre-pre-amplifier in an SGC. In some embodiments, the SGC provided herein comprises additional components such an amplifier, a pre-amplifier, and/or a pre-pre-amplifier.

In some embodiments, the compositions of the present disclosure include an “amplifier,” which is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides described herein, the amplifier can include modified nucleotides and/or nonstandard inter-nucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated herein by reference in their entirety.

In some embodiments, the compositions of the present disclosure include a “pre-amplifier,” which is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated herein by reference in its entirety.

In some embodiments, the compositions of the present disclosure include a “pre-pre-amplifier.” which is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated herein by reference in its entirety.

In some embodiments, the target is a protein. In such embodiments, the target protein can be detected using IHC/ICC (described further herein below). In such embodiments, the probe is a primary antibody that binds to the target protein. In some embodiments, the compositions further comprise a secondary antibody that binds to the primary antibody, wherein the secondary antibody binds to a detectable label.

In some embodiments, such as when the composition is being used for detecting both a target nucleic acid and a target protein, the composition comprises a combination of probes described above (e.g., a probe comprising a T section and an L section, and a primary antibody, and other components as described above).

As described above, the compositions include various probes that include a detectable label. As used herein, a “label” or a “detectable label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In some embodiments, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, quantum dots, dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the present disclosure, including detectable enzyme/substrate combinations (Pierce, Rockford IL; Santa Cruz Biotechnology, Dallas TX; Life Technologies, Carlsbad CA). In one embodiment of the disclosure, the enzyme can utilize a chromogenic or fluorogenic substrate to produce a detectable signal, as described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. In some embodiments, useful detectable signals are chromogenic or fluorogenic signals. Accordingly, enzymes that are suitable for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactosidase 6-phosphogalactohydrolase, β-glucosidase, aglucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are well known to those skilled in the art based on the present disclosure and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl−1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and Quintolet for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in U.S. Patent publication 2012/0100540, which is incorporated herein by reference in its entirety. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb ¹⁷²Yb, ¹⁷³Yb, ¹⁷⁴Yb, ¹⁷⁵Lu, and ¹⁷⁶Yb. Metal isotopes can be detected, for example, using time-of-flight mass spectrometry (TOF-MS)(for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540, which is incorporated herein by reference in its entirety.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). For example, in some embodiments, the detectable label is a fluorophore selected from a fluorescein, a rhodamine, a boron-dipyrromethene, a cyanine, an oxazine, a thiazine, a coumarin, a naphthalimide, a rhodol, a naphthalene, a squarane, a porphyrin, and a flavin. Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa Fluor dyes (Molecular Probes), such as Alexa Fluor 488, Alexa Fluor 750, and the like; 4,4-difluoro-4-bora-3a,4a-diaza-s-indacene (BODIPY®) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), DyLight 395XL, DyLight 550, DyLight 650, ATTO 390, ATTO 425, ATTO 465, ATTO 488, ATTO 490LS, ATTO 495, ATTO 514, ATTO 520, ATTO 532, ATTO Rho6G, ATTO 542, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

The compositions further include a quinone compound, which is the element of the composition that reduces autofluorescence in the sample. While a variety of quinone compounds can be used in the compositions, in some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone.

In the compositions disclosed herein, the target (e.g., the target nucleic acid or the target protein) is present in a biological sample, and thus the composition further comprises other components of the biological sample. In some embodiments, the biological sample is a tissue specimen or is derived from a tissue specimen. In some embodiments, the sample is a formalin fixed paraffin embedded (FFPE) tissue specimen, a fixed frozen tissue specimen, or a fresh frozen tissue specimen. In particular embodiments, the biological sample is a FFPE tissue specimen. In some embodiments, the biological sample is a fixed frozen tissue specimen prepared with a fixative other than formalin, such as ethanol, methanol, Bouin's, B5, or I.B.F. In some embodiments, the biological sample is a cytological sample or is derived from a cytological sample. Other exemplary biological samples include a cell culture, including a primary cell culture, a cell line, a cell suspension, an organ, an organelle, a biological fluid, and the like. Additional biological samples include but are not limited to a skin sample, tissue biopsies, including fine needle aspirates, stool, bodily fluids, including blood and/or serum samples, saliva, semen, and the like.

### 3. METHODS OF DETECTION

In addition to compositions, also disclosed herein are methods of detecting a target in a biological sample. The methods comprise: contacting the sample with a probe that binds to the target; contacting the sample with a quinone compound; and detecting a signal corresponding to the target in the sample. The use of the quinone compound in the method reduces autofluorescence in the biological sample, allowing for the methods to provide improved detection of signals corresponding to the target in the sample.

In some embodiments, the target in the biological sample is a nucleic acid. Methods for in situ detection of nucleic acids are well known to those skilled in the art (see, for example: US 2008/0038725; US 2009/0081688; and Hicks et al., J. Mol. Histol. 35:595-601 (2004)). As used herein, in situ hybridization” or “ISH” refers to a type of hybridization that uses a directly or indirectly labeled complementary DNA or RNA strand, such as a probe, to bind to and localize a specific nucleic acid in a sample, in particular a portion or section of tissue or cells (in situ). The probe types can be double stranded DNA (dsDNA), single stranded DNA (ssDNA), single stranded complimentary RNA (sscRNA), messenger RNA (mRNA), microRNA (miRNA), ribosomal RNA, mitochondrial RNA, and/or synthetic oligonucleotides.

In some such embodiments, the target is RNA, such as messenger RNA (mRNA), microRNA (miRNA), ribosomal RNA (rRNA), mitochondrial RNA, or non-coding RNA (e.g., long non-coding RNA (lncRNA)). Embodiments of the methods provided herein include methods of detecting a target RNA using in situ hybridization (ISH). RNA in situ hybridization (ISH) is a molecular biology technique widely used to measure and localize specific RNA sequences (mRNAs, lncRNAs, and miRNAs) within cells such as circulating tumor cells (CTCs) or tissue sections while preserving the cellular and tissue context. RNA ISH therefore enables spatial-temporal visualization as well as quantification of gene expression within cells and tissues. It has wide applications in research and in diagnostics. RNA ISH signals are dot-like, and each dot represents a single molecular of RNA. Therefore, ISH signals in general are susceptible to tissue autofluorescence. Highly multiplexed ISH assays are also increasingly desirable in the era of multi-omics research, which when combined with effective autofluorescence removal, can be significantly enhanced.

In one embodiment, the RNA ISH (RISH) used herein to detect mRNA targets is RNAscope®, which is described in more detail in, e.g., U.S. Pat. Nos. 7,709,198, 8,604,182, and 8,951,726, which are herein incorporated by reference in their entireties. Specifically, RNAscope® describes using specially designed oligonucleotide probes, sometimes referred to as “double-Z” or ZZ probes, in combination with a branched-DNA-like signal amplification system to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)). Such a probe design greatly improves the specificity of signal amplification because only when both probes in each pair bind to their intended target can signal amplification occur. Specific details of such probe designs, including target probes (e.g., probes comprising a T section and an L section), and signal-generating complexes (e.g., comprising a label probe and one or more of an amplifier, a pre-amplifier, and a pre-pre-amplifier) are discussed above in the compositions section; any such probes and any combination thereof can be used in the methods of detecting a nucleic acid disclosed herein.

In one embodiment, the RNA ISH (RISH) used herein to detect mRNA targets is BaseScope™, which is described in more detail in, e.g., US Patent Publication No. 2017/0101672, which is incorporated herein by reference in its entirety. Specifically, BaseScope™ includes the use of specially designed oligonucleotide probes, sometimes referred to as “double-Z” or ZZ probes, in combination with a branched-DNA-like signal amplification system to reliably detect target RNA with single-molecule sensitivity under standard bright-field microscopy. The BaseScope® platform enables applications such as the detection of exon junctions/splice variants, short/highly homologous RNA sequences (50-300 bases), and point mutations at single cell sensitivity (Anderson, C. M. et al. Visualizing Genetic Variants, Short Targets, and Point Mutations in the Morphological Tissue Context with an RNA In Situ Hybridization Assay. 0.1 Vis. Exp. (2018); doi:10.3791/58097). Such a probe design greatly improves the specificity of signal amplification because only when both probes in each pair bind to their intended target can signal amplification occur, enabling the detecting of biological events in cells and in situ using a single Z probe pair. Again, specific details of probe designs that can be used in such methods are described above in the compositions section; any such probes and any combination thereof can be used in the methods of detecting a nucleic acid disclosed herein.

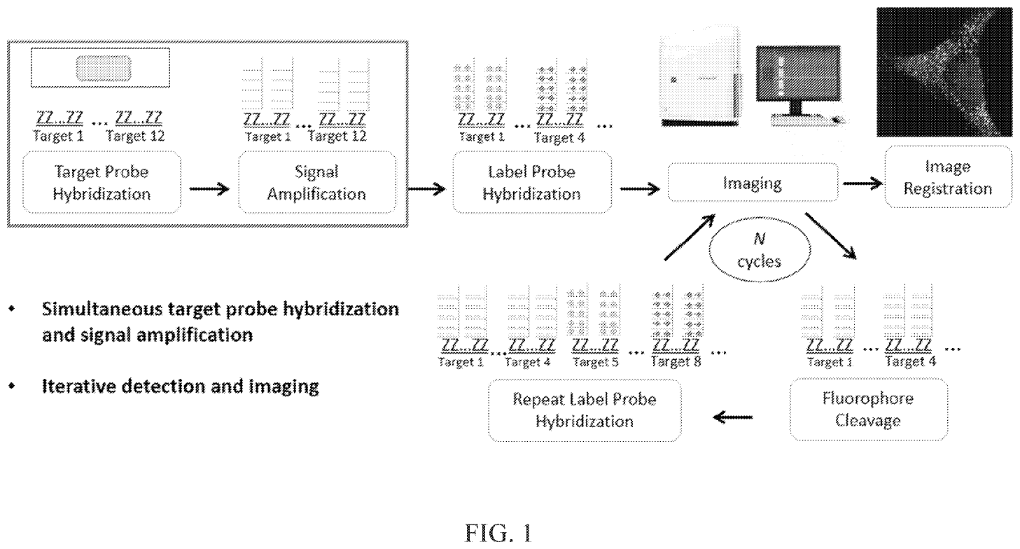

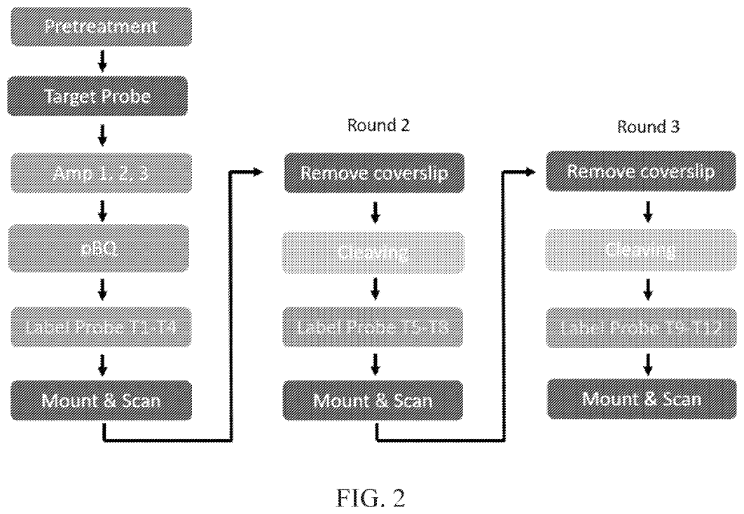

In another embodiment, the RNA ISH (RISH) used herein to detect mRNA targets is a multiplex assay, such as RNAscope® HiPlex, which is described in more detail in, e.g., International Patent Application Publication No. WO 2020/168162, which is incorporated herein by reference in its entirety. Specifically, RNAscopc® HiPlex allows simultaneous detection of up to 12 different RNA targets per slide-mounted sample, enabled by employing cleavable fluorophores and iterative detection. An exemplary schematic is shown in FIG. 1. The RNAscope® double Z probe designs and branched-DNA-like signal amplification method were adapted, such that 12 target sequences are simultaneously hybridized with double Z probes that bind to 12 orthogonal signal amplification systems (indicated by the different colored label probes in FIG. 1). In the embodiment depicted, the first four targets are detected via four non-spectral overlapping cleavable fluorescent dye-conjugated oligos (label probes) and imaged using a conventional fluorescent microscope or scanner. Fluorophores are then cleaved off from the label probes and the next four targets are labeled and imaged using the same method. After three rounds of detection of four targets each, images are registered using an image registration software algorithm to create the final composite of superimposed images with single-cell resolution. An exemplary workflow in which application of a quinone compound (e.g., p-benzoquinone (pBQ)) is incorporated into a RNAscope® HiPlex assay is shown in FIG. 2. In this exemplary workflow, p-benzoquinone is applied after the set of amplifiers of the SGC (“Amp 1, 2, 3”), but before the application of a label probe.

In another embodiment, the RNA ISH methods of the present disclosure include the use of probes that form stable DNA hairpins, along with a DNA initiator probe. These probes can be used to detect a target mRNA using a hybridization chain reaction (HCR) mechanism. The addition of an initiator strand of DNA to the stable mixture of two hairpin species triggers a chain reaction of hybridization events between the hairpins, which is used to amplify a detectable signal (see, e.g., Dirks, R. M. and Pierce, N. A. Triggered amplification by hybridization chain reaction. Proc. Natl. Acad Sci. USA 101, 15275-15278 (2004)).

In some such embodiments, the target is DNA. Embodiments of the methods provided herein include methods of detecting a target DNA using in situ hybridization (ISH). In some specific embodiments, the DNA ISH used herein is based on RNAscope®. It uses the RNAscope core technology, with modifications added in the “pretreatment” steps to optimize for DNA detection. Specifically, in some embodiments, a DNA denaturation step using formamide at an elevated temperature (e.g., 70% formamide at 80° C.) is added before probe hybridization, which is described in more detail in, e.g., U.S. Pat. Nos. 7,709,198, 8,604,182, and 8,951,726, which are incorporated herein by reference in their entireties. Use of RNAscope® based methods to detect DNA in a sample can further involve removal of RNA from the sample. Methods for removing RNA are known in the art; one method is disclosed in U.S. Provisional Patent Application No. 63/080,398, which is incorporated herein by reference. In one embodiment, the DNA ISH used herein is based on BaseScope®, which is discussed above. In another embodiment, the DNA ISH used herein is described in International Patent Application Publication No. WO 2020/185846. This assay uses a probe design strategy that provides specific detection of double stranded DNA using the principles of RNAscope-, where each probe pair binds to both strands of the double stranded DNA. Like RNAscope® probes, each probe contains a sequence segment that binds to a specific sequence in the target. For double stranded nucleic acid detection, for example, DNA detection, two probes bind to adjacent sites on opposite strands in the target double stranded nucleic acid. Only when both probes bind to their respective target sites simultaneously can a full binding site for the signal amplification molecule (for example, a pre-amplifier or a pre-pre-amplifier) be formed, leading to successful signal amplification and detection. The target DNA has any suitable length, from about 1 kilobase (kb) to hundreds of kb or even larger (e.g., a full chromosome).

In some embodiments, the target is a protein. In some embodiments, the probe is a primary antibody that binds to the protein target, and the method further comprises contacting the sample with a secondary antibody that binds to the primary antibody, wherein the secondary antibody binds to a detectable label. In embodiments in which the target is a protein, the detecting methods can involve immunocytochemistry (ICC)/immunohistochemistry (IHC).

Like ISH in that temporal and spatial information can be preserved, IHC/ICC is another widely used technique, but for detecting target proteins. Taking advantage of specificity of antigen-antibody binding, IHC facilitates the visualization of the high-resolution distribution and localization of specific target proteins within cells and within their proper histological context. In some embodiments, IHC/ICC comprises preparing a whole-cell preparation, a paraffin-embedded tissue, a frozen tissue, or a floating section. If it is a paraffin-embedded tissue that is prepared, the next step for IHC is de-paraffinization to remove paraffin. During the tissue preparation and preservation process, if a crosslinking agent, such as formalin, is used, formalin fixation may mask epitopes and result in decreased immunoreactivity (see Arnold et al., Biotech Histochem 71:224-230 (1996)). Formalin fixation is a time-dependent process in which increased fixation time results in continued formaldehyde group binding to proteins to a point of equilibrium (see Fox et al., J Histochem Cytochem 33:845-853 (1985)). Studies have shown that formalin fixation, especially if prolonged, results in decreased antigenicity (see Battifora and Kopinski, J Histochem Cytochem 34:1095-1100(1986)), which limits the use of formalin-fixed tissues for diagnostic IHC (see Ramos-Vara, Vet Pathol 42:405-426 (2005), Webster et al., J Histochem Cytochem. 57(8): 753-761 (2009)).

As disclosed herein, the methods provided herein can utilize concurrent detection of multiple targets, e.g., multiple nucleic acids, multiple proteins, or one or more nucleic acids and one or more proteins. For example, in some embodiments, the method can include incubating a biological sample with a primary antibody, treating the biological sample with a crosslinking agent, and detecting the target nucleic acid by in situ hybridization. In some embodiments, the method further comprises treating the biological sample with a protease after treating the biological sample with the crosslinking agent and before detecting the target nucleic acid by in situ hybridization. In other embodiments, the method further includes incubating the biological sample with a secondary antibody or other labeling methods after detecting the target nucleic acid by in situ hybridization. In some embodiments, the preparation methods include performing the following steps sequentially: incubating the biological sample with a primary antibody; treating the biological sample with a crosslinking agent; treating the biological sample with a protease; detecting the target nucleic acid by ISH; and incubating the biological sample with a secondary antibody or other labeling methods to detect a peptide, polypeptide, or protein of interest (e.g., using ICC, IHC, and IF). In some embodiments, additional steps may be included. The benefits of simultaneously detecting a target nucleic acid using ISH and a target protein using IHC/ICC can be critical assessing a biological sample and/or making a treatment or diagnostic decision. For example, simultaneously detecting a target nucleic acid using ISH and a target protein using IHC/ICC increases the throughput of analysis and reduces the burden of time and cost associated with the investigation of those individual components. Furthermore, combined detection in the same sample offers researchers and medical professionals information which cannot be gained from staining on separate sections, such as, visualizing both a secreted protein and the origin of its cell(s), and interaction of distinct cell types. Though the steps in both techniques described above appear similar, it has been problematic to apply the two techniques to one sample, and accurately imaging the results of these assays has been even more challenging. Thus, as described further below, the image processing methods of the present disclosure facilitate the robust acquisition of qualitative and quantitative information of various targets-of-interest detected simultaneously in a biological sample using in situ hybridization and IHC/ICC.

Whether the target is a nucleic acid or a protein, or a combination thereof, the methods include a step of contacting the sample with a quinone compound. As demonstrated herein, application of a quinone compound to a biological sample reduces autofluorescence in the sample, and thus results in improved detection of signals from the targets in the sample. A variety of quinone compounds can be used in the methods; in some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone.

In some embodiments, the method comprises a step of contacting the sample with the quinone compound in an amount and for a period of time effective to reduce autofluorescence in the sample, in order to improve the detection of the target(s) in the sample. In some embodiments, the method comprises contacting the sample with a composition comprising the quinone compound at a concentration of about 1 mM to about 100 mM, or about 10 mM to about 150 mM, e.g., about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some embodiments, the method comprises contacting the sample with the quinone compound for about 5 minutes to about 24 hours, or about 5 minutes to about 12 hours, or 5 minutes to about 60 minutes, e.g., about 10 minutes to about 45 minutes, e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 1 hour, about 2 hours, about 3 hours, about 4 hours, about 5 hours, about 6 hours, about 7 hours, about 8 hours, about 9 hours, about 10 hours, about II hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the methods further comprise a step of washing the sample after it is contacted with the quinone compound. In some embodiments, the sample is washed after it is contacted with the quinone compound, but before any detectable label or label probe is contacted with the sample. The sample can be washed, e.g., with a buffer that is being used for the probe hybridizations. In some embodiments the sample can be washed with a saline-sodium citrate buffer (SSC).

The methods further comprise a step of detecting a signal corresponding to the target in the sample. The specific detecting method will depend on the specific label being used. Labels are generally discussed above in the compositions section. Well-known methods such as microscopy, cytometry (e.g., mass cytometry, cytometry by time of flight (CyTOF), flow cytometry), or spectroscopy can be utilized to detect/visualize chromogenic, fluorescent, or metal detectable signals associated with the respective targets. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of targets in the same sample.

In the case of using fluorophores as labels, the fluorophores to be used for detection of multiple targets are selected so that each of the fluorophores are distinguishable and can be detected concurrently in the fluorescence microscope in the case of concurrent detection of targets. Such fluorophores are selected to have spectral separation of the emissions so that distinct labeling of the targets can be detected concurrently. Methods of selecting suitable distinguishable fluorophores for use in methods of the disclosure are well known in the art (see, for example, Johnson and Spence, “Molecular Probes Handbook, a Guide to Fluorescent Probes and Labeling Technologies, 11th ed., Life Technologies (2010)).

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of targets. Generally, the labels are conjugated to the label probe by a chemical linker that is cleavable. Methods of conjugating a label to a label probe so that the label is cleavable are well known to those skilled in the art (see, for example, Hermanson, Bioconjugate Techniques, Academic Press, San Diego (1996); Daniel et al., BioTechniques 24(3):484-489 (1998)). One system of labeling oligonucleotides is the FastTag™ system (Daniel et al., supra, 1998; Vector Laboratories, Burlinghame CA). Various cleavable moieties can be included in the linker so that the label can be cleaved from the label probe. Such cleavable moieties include groups that can be chemically, photochemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xₐ (cleavage site IEGR/), tobacco etch virus protease (cleavage site ENLYFQ/G), and thrombin (cleavage site LVPR/GS) (see, for example, Oxford Genetics, Oxford. UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad Sci. USA 113:11046-11051 (2016)). In one embodiment, TCEP is used as an agent to cleave a label from a label probe.

For methods of the present disclosure for in situ detection of targets in a cell, the cell is optionally fixed and/or permeabilized before hybridization of the target probes. Fixing and permeabilizing cells can facilitate retaining the targets in the cell and permit the target probes, label probes, and so forth, to enter the cell and reach the target molecule. The cell is optionally washed to remove materials not captured to a target. The cell can be washed after any of various steps, for example, after hybridization of the probes to the targets to remove unbound target probes, and the like. Methods for fixing and permeabilizing cells for in situ detection of targets (e.g., nucleic acids), as well as methods for hybridizing, washing and detecting targets (e.g., nucleic acids), are also well known in the art (see, for example. US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss. New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, CA), and enzymes (for example, lysozyme, lipases, proteases and peptidases). Permeabilization can also occur by mechanical disruption, such as in tissue slices.

In situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a target that is a biomarker indicative of a pathology.

It is understood by those skilled in the art based on the present disclosure that any of a number of suitable samples can be used for detecting targets using methods provided herein. The sample for use in methods provided herein will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions and/or cells isolated from an organism such as a mammal. Exemplary biological samples include, but are not limited to, a cell culture, including a primary cell culture, a cell suspension, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, for example, Dey, “Cytology Sample Procurement, Fixation and Processing” in Basic and Advanced Laboratory Techniques in Histopathology and Cytology pp. 121-132, Springer, Singapore (2018). “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithehal Neoplasia: A Beginner's Manual, Sellors and Sankaranarayanan, eds., International Agency for Research on Cancer, Lyon, France (2003); Kalaf and Cooper, J. Clin. Pathol. 60:449455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In some embodiments, the sample is a tissue specimen or is derived from a tissue specimen. In some embodiments, the tissue specimen is formalin-fixed paraffin-embedded (FFPE). In some embodiments, the tissue specimen is fresh frozen. In some embodiments, the tissue specimen is prepared with a fixative other than formalin. In some embodiments, the fixative other than formalin is selected from the group consisting of ethanol, methanol, Bouin's, B5, and I.B.F. In other embodiments, the sample is a blood sample or is derived from a blood sample. In still other embodiments, the sample is a cytological sample or is derived from a cytological sample.

When the sample is a FFPE tissue specimen, the method may further comprise a deparaffinization step (also known as dewaxing) prior to contacting the sample with any probes, the quinone compound, and other components. Deparaffinization is typically performed washing the specimen with a non-polar solvent, such as xylene, a mineral oil, or other suitable hydrocarbon-based solvent. The washing step with the non-polar solvent is typically performed multiple times. An optional heating step to melt the wax can be performed prior to washing. After the washing steps with the non-polar solvent, the non-polar solvent can be removed by successive washing steps with graded concentrations of ethanol, e.g., first with a 50:50 mixture of xylene and ethanol, followed by washing with solutions having successively lower concentrations of ethanol (e.g., 100% ethanol, then one or more washes with solutions of 95% ethanol, 90% ethanol, 85% ethanol, 80% ethanol, 75% ethanol, 70% ethanol, 65% ethanol, 60% ethanol, 55% ethanol, and/or 50% ethanol, or any combination thereof), followed by one or more final washes with water.

As would be understood by one of ordinary skill in the art based on the present disclosure, embodiments of the compositions and methods provided herein include the ability to measure and/or quantify a detectable label. In some embodiments, the label will be detected using a single-plex format, and in other embodiments, the label will be detected in a duplex or multiplex format, which facilitates the detection and/or quantification of more than one target. In accordance with these embodiments, the methods and compositions described herein include the use of detection/quantification systems, such as the use of computer software and hardware. In one embodiment, a detection/quantification system of the present disclosure includes a computer and suitable software for receiving user instructions, either in the form of user input into a set of parameter fields (e.g., in a GUI, or in the form of preprogrammed instructions, which can be preprogrammed for a variety of different specific operations to assess a sample). For example, the software can be preprogrammed for one or more operation such as sample handling, slide handling, de-paraffinization, de-crosslinking, hybridization, washing, and the like, as described herein. The software can convert these instructions to appropriate language for controlling the operation of components of the system (e.g., for controlling a fluid handling element and/or laser). The computer can also receive data from other components of the system (e.g., from a detector), and can interpret and/or process the data, provide it to a user in a readable format, or use that data to initiate further operations, in accordance with any programming by the user. In this manner, the system can, for example, quantify any number of detectable labels, compare them to each other (e.g., in a multiplex format) or to controls (e.g., a reference control), and generate a value corresponding to the amount of mRNA in the sample. Any appropriate computer software can be used to facilitate quantitation/detection of a label, including but not limited to, ImageJ, QuPath, and other commercial software such as HALO and VsioPharm.

In some embodiments, the detection/quantification system can measure or quantify target levels in a sample from a subject suspected of having a disease or condition, and compare those levels to a reference control (e.g., a healthy control sample) in order to determine whether the amount of a given target with respect to control levels supports a diagnosis that the subject has that disease or condition. In some embodiments, targets can be compared to each other to determine whether a subject has a disease or condition. In some embodiments, the system can include means for determining proportions or ratios of target levels with respect to each other and to controls. As would be recognized by one of ordinary skill in the art based on the present disclosure, reference levels or reference controls can be obtained from various sources, including but not limited to, databases of target levels, patient look-up tables, and/or directly from patient samples; which source is used for a given assessment depends on various factors, such as the target being evaluated, the disease context, the cell/tissue type, and the like.

In some embodiments, the system enables a user (e.g., medical processional such as a pathologist) to view a sample from a subject and compare it to a reference control (from the same subject or a different subject (e.g., healthy control sample). In accordance with these embodiments, the system enables a user to make a determination as to whether a target is present in sufficient amounts to warrant a diagnosis that the subject has or is likely to develop a disease condition. The system can also provide means for assessing spatial patterns of target expression and/or determine whether a target is more or less abundant in a spatially-restricted area of a sample that may correspond to a meaningful anatomical feature (e.g., a tumor micro-environment).

### 4. KITS

Embodiments of the present disclosure also include a kit for detecting a target in a biological sample. In accordance with these embodiments, the kit includes a probe that binds to the target, a quinone compound, and instructions for conducting an assay to detect the target in the biological sample. The inclusion of the quinone compound allows for the reduction of autofluorescence in the biological sample, such that the kit can provide improved detection of the target in the sample.

In some embodiments, the kit is for detecting a nucleic acid target. In such embodiments, the kit comprises probes and other elements (e.g., SGCs) useful for detecting such targets, as disclosed elsewhere herein. For example, in some embodiments, the probe is a first target probe comprising a T section and an L section, wherein the T section is complementary to a portion of the nucleic acid target and wherein the L section is complementary to a nucleic acid component of a signal generating complex. In some embodiments, the kit further comprises a second target probe comprising a T section and an L section, wherein the T section is complementary to a portion of the nucleic acid target and wherein the L section is complementary to a nucleic acid component of a signal generating complex. In some embodiments, the T sections of the first target probe and the second target probe are complementary to non-overlapping regions of the nucleic acid target. In some embodiments, the L sections of the first target probe and the second target probe are complementary to non-overlapping regions of the signal generating complex.

In some embodiments, the kit further comprises a signal generating complex comprising at least one detectable label. In some embodiments, the signal generating complex comprises a label probe that binds to the detectable label, and optionally, one or more of an amplifier, a pre-amplifier, and a pre-pre-amplifier. In some embodiments, the signal generating complex comprises a label probe, an amplifier, and a pre-amplifier. In some embodiments, the detectable label is a fluorophore or a quantum dot. In some embodiments, the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

In some embodiments, the nucleic acid target is selected from an RNA and a DNA. In some embodiments, the nucleic acid target is an RNA, and the RNA is selected from messenger RNA (mRNA), microRNA (miRNA), ribosomal RNA (rRNA), mitochondrial RNA, and non-coding RNA. In some embodiments, the nucleic acid target is mRNA. In some embodiments, the nucleic acid target is DNA.

In some embodiments, the kit is for detecting a protein target. In some embodiments, the probe is a primary antibody that binds to the protein target, and the kit further comprises a secondary antibody that binds to the primary antibody, wherein the secondary antibody binds to a detectable label.

In some embodiments, the kit further comprises a second probe for a second target in the biological sample. In some embodiments, the second target is a nucleic acid or a protein.

In some embodiments, the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone. In some embodiments, the quinone compound is p-benzoquinone or hydroquinone. In some embodiments, the quinone compound is p-benzoquinone. In some embodiments, the quinone compound is hydroquinone. In some embodiments, the kit comprises a composition of the quinone compound at a concentration of about 1 mM to about 100 mM, or about 10 mM to about 150 mM, e.g., about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM.

In some embodiments, the kit further includes other agents or materials for performing in situ hybridization or hybridization chain reaction assays, including but not limited to, fixing agents and agents for treating samples for preparing hybridization, agents for washing samples, and the like. In some embodiments, the kit includes at least one of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof. In some embodiments, the kit provided herein comprises agents for performing RNAscope®, BaseScoper™, or RNAscope® HiPlex, which are described above.

The kits of the present disclosure may further include instructions and/or packaging material, which generally includes to a physical container for housing and/or delivering the components of the kit. The packaging material can maintain sterility of the components, and can be made of material commonly used for such purposes (e.g., paper, corrugated fiber, glass, plastic, foil, ampules, vials, tubes, etc.).

Kits provided herein can include labels or inserts, such as instructions for performing an assay. Labels or inserts can include “printed matter.” e.g., paper or cardboard, separate or affixed to a component, a kit or packing material (e.g., a box), or attached to, for example, an ampule, tube or vial containing a kit component. Labels or inserts can additionally include a computer readable medium, such as a disk (e.g., hard disk, card, and memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

### 5. IMAGE PROCESSING

In some embodiments, the compositions, methods, and kits of the disclosure an further include a method for enhancing detection of a target. The method includes imaging a sample comprising a target signal to create a probe image (e.g., a signal from a target disclosed herein, which is from a sample to which a quinone compound has been applied to reduce autofluorescence), and imaging the sample comprising no target signal to create a background image. The method further includes modifying the background image to create an adjusted background image based on at least one image metric, and subtracting the adjusted background image from the probe image to create a final image comprising an enhanced target signal.

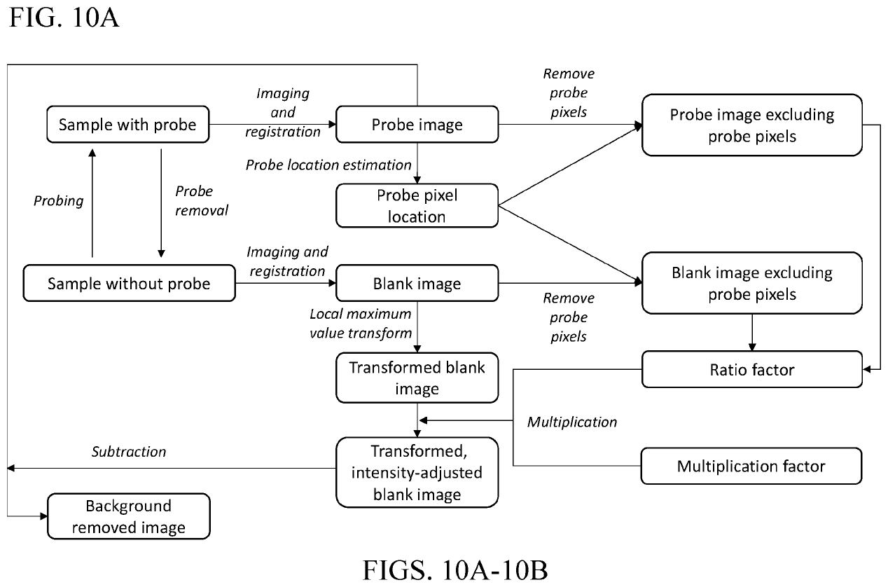

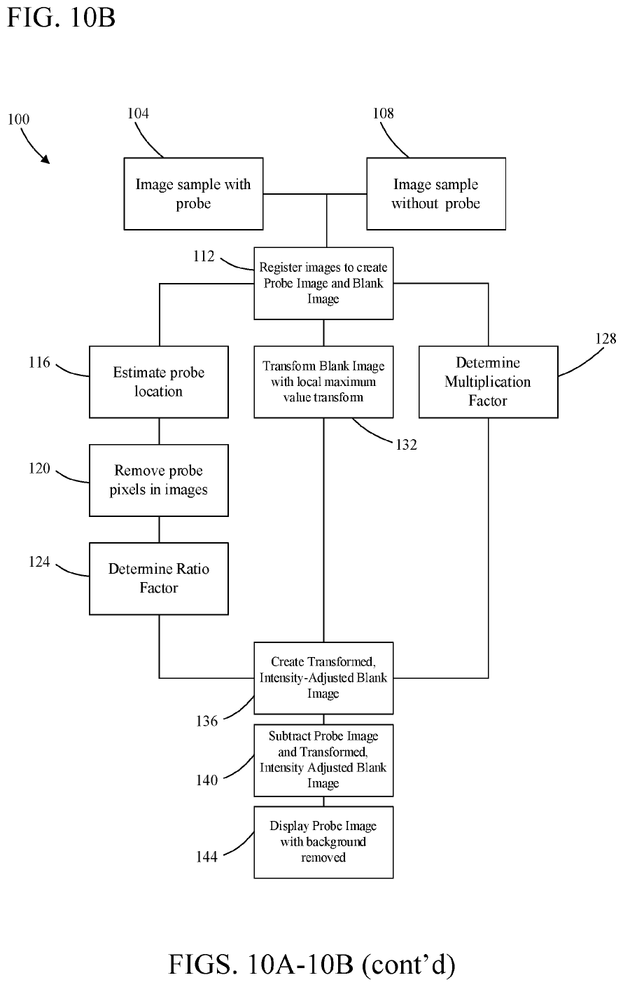

Accordingly, embodiments of the present disclosure include a method 100 for enhancing detection of a target. In some embodiments, the method 100 includes an image processing method. The method 100 is illustrated in FIG. 10B as a flowchart of steps, whereas FIG. 10A illustrates a plurality of images and corresponding factors used in the method 100 to modify the images. In the illustrated embodiment, the method 100 is implemented at least in part with a computer having corresponding instructions stored on a memory (i.e., a non-transitory computer readable medium). The final images, and in some embodiments the intermediate images, from the method 100 are stored in a memory. In some embodiments, the memory is accessible by a network. In some embodiments, user input or instructions are receivable or accessible over the network.

The method 100 includes imaging a sample with a target signal (STEP 104) to create a probe image and imaging a sample with no target signal (STEP 108) to create a background image (i.e., blank image”). In some embodiments, the imaging utilizes a fluorescent microscope coupled to a computer via a network. In some embodiments, the target signal is obtained by subjecting the sample to a fluorescent in situ hybridization assay and/or an immunofluorescence assay. In some embodiments, the background image with no target signal is obtained by removing the target signal from the sample (i.e., by a cleaving process). In other embodiments, the background image with no target signal is obtained before the assay is performed. In other words, in some embodiments STEP 104 occurs before STEP 108 and in other embodiments STEP 104 occurs after STEP 108. In some embodiments, the target signal comprises a fluorescent label bound to a target nucleic acid. In other embodiments, the target signal comprises a fluorescent label bound to a target peptide or polypeptide.

With continued reference to FIG. 10B, the method 100 includes a STEP 112 of registering the probe image and the background image. Potential background fluorescence discrepancy between the probe image and the background image create spatial pattern mismatches that occur due to whole sample movement between different rounds of image acquisition. To remove such discrepancies, image registration techniques (e.g., phase correlation) are utilized. Robust image registration (e.g., STEP 112) utilized detection and matching of image features to compensate for any global sample movement (i.e., translation and rotation).

The method 100 further includes modifying the background image (STEP 136) to create an adjusted background image (e.g., transformed, intensity-adjusted blank image) based on at least one image metric. As explained further herein, the at least one image metric is a ratio factor (STEPS 116, 120, 124), a multiplication factor (STEP 128), a local maximum value transform (STEP 132), and any other suitable metric. In some embodiments, the method 100 includes a single image metric. In other embodiments, the method 100 includes a combination of image metrics.

With continued reference to FIG. 10B, the method 100 further includes subtracting the adjusted background image from the probe image (STEP 140) to create a final image comprising an enhanced target signal. In other words, the modified (i.e., transformed, adjusted, scaled, etc.) blank image is used in the subtracting step (STEP 140) instead of the original blank image. In some embodiments, the method 100 includes displaying the final image (STEP 144) on a display (e.g., a computer display). The final image may be saved to a memory and may be accessible by a user, for example, over a network. As such, the method 100 provides improved signal detection in the presence of a background with tissue autofluorescence.

In some embodiments, the image metric is a ratio factor to account for intensity differences in background between the blank image and the probe image. Intensity differences can occur when image acquisition settings are different or from photobleaching during fluorophore excitation. To compensate for background intensity differences, the method 100 includes STEPS 116, 120, and 124 to determine a ratio factor that compares the overall background intensity of the probe image versus the blank image. First, the pixel locations of the probe are estimated (STEP 116). The probe locations in the probe image are estimated using, for example, the White Top Hat algorithm (Gonzalez & Woods, 2008, Digital Image Processing), bandpass filtering (Shenoi, 2006, Introduction to Digital Signal Processing and Filter Design), or any combination of suitable methods. After determining an estimated location of the target signals in the probe image (STEP 116), the pixels at the estimated probe locations are excluded from both the probe image and the blank image (STEP 120), resulting in background-pixel-only images (i.e., background-only images). In other words, STEP 120 includes removing the estimated location from the probe image to create a first background-only image and removing the estimated location from the blank image (background image) to create a second background-only image.

Following removal of the estimated probe locations from both images (STEP 120), the method 100 includes a STEP 124 to determine a ratio factor. In other words, a statistical metric for both the probe-excluded blank image and the probe-excluded probe image is evaluated and incorporated into a ratio factor. As explained further herein, the ratio factor is utilized in some embodiments to modify the background image to create an adjusted background image (STEP 136). In other words, modifying the background image to create an adjusted background image can include, in some embodiments, scaling the background image by the ratio factor.

In some embodiments, the at least one image metric is a ratio factor of the first background-only image and the second background-only image. For example, the ratio factor in some embodiments is a first intensity to a second intensity, with the first intensity is determined from the first background-only image and the second intensity is determined form the second background-only image. In some embodiments, the first and second intensities used in the ratio factor are statistical metrics such as a statistical mean, median, or a combination of both for any portion of (including all) the intensity values in an image.

In some embodiments, the first intensity is the mean of a plurality of pixel intensity values in the first background-only image and the second intensity is the mean of a plurality of pixel intensity values in the second background-only image. In some embodiments, the mean is of all the pixel intensity values in the image. In other embodiments, the first intensity is the median of a plurality of pixel intensity values in the first background-only image, and the second intensity is the median of a plurality of pixel intensity values in the second background-only image. In some embodiments, the median is of all the pixel intensity values in the image. In another embodiment, the first intensity is the mean of a central approximately 80% of all the pixel intensity values (i.e., excluding the approximate top 10% and the approximate bottom 10%) in the first background-only image, and the second intensity is the mean of a central approximately 80% of all the pixel intensity values in the second background-only image.

In some embodiments, the image metric is a multiplication factor to account for potential local intensity differences between the blank image and the probe image. In particular, the method 100 in the illustrated embodiment includes STEP 128 to determine the multiplication factor. In some embodiments, the multiplication factor is within a range of approximately 1.0 to approximately 1.2. In other embodiments, the multiplication factor is within a range of approximately 1.0 to approximately 1.1. As explained further herein, the multiplication factor is utilized in some embodiments to modify the background image to create an adjusted background image (STEP 136). In other words, modifying the background image to create an adjusted background image can include, in some embodiments, scaling the background image by the multiplication factor.

In some embodiments, the image metric is a local maximum value transform. In particular, the method 100 in the illustrated embodiment includes STEP 132 to transform the blank image with a local maximum value transform. Even after global image registration, there may remain local background pattern mismatches that from, for example, image acquisition at different focal planes, or samples not firmly attached to the supporting material (e.g., glass slides) and partially moving between imaging sessions. To resolve this issue, local mismatches are compensated with a transform. In the illustrated embodiment, for each pixel in the blank image (“pixel of interest”), a neighborhood of a pre-defined radius surrounding the pixel of interest is searched. The search process will find the pixel of maximum intensity, and this maximum intensity is assigned to that pixel of interest. This searching procedure is performed for each pixel of interest, searching its neighborhood in the original blank image, to form a transformed blank image. As explained in greater detail herein, the transformed blank image can be used instead of the original blank image in the later subtracting step (i.e., STEP 140). In some embodiments, the pre-defined radius (“match distance”) is adjustable.

In some embodiments, the pre-defined radius used in the local maximum valve transform is within a range of approximately 0 to approximately 5 pixels. In other words, the local maximum value transform includes a search radius within a range of approximately 0 to 5 pixels. A pre-defined radius of 0 pixels is utilized, for example, when there is no noticeable local background pattern mismatch. In some embodiments, the search area is simplified to reduce computational time by using eight angularly equally spaced lines (i.e., 45 degrees apart), each with a single-pixel width, radiating from the pixel of interest.

In some embodiments, the image metric is a block-matching transform. In particular, the method 100, in some embodiments, includes a step to transform the blank image with a block-matching transform. In some embodiments, the block-matching transform is used in place of the local maximum value transform to resolve the issue of local mismatches. In some embodiments, a block (“block of interest”) is used with a pre-defined block size (e.g., a 3-pixel-by-3-pixel block). Each block in the blank image is compared with blocks of the same size in the probe image in nearby locations (i.e., within a pre-defined block search size). The search determines the nearby block that is most similar to the block of interest. A similarity metric is utilized to measure the similarity of the blocks, and the searched nearby block with the highest similarity metric is determined to be the target block. Then, the block of interest is moved to the corresponding location of the target block. In some embodiments, the similarity metric is a mean absolute difference, a sum of absolute difference, a mean squared difference, or a sum of squared difference, wherein the differences are the pixel intensity differences between the two blocks being compared. As such, the block-matching transform is performed for each block of interest, searching its corresponding neighborhood in the probe image and moving its location accordingly, to form a transformed blank image. In some embodiments, this transformed blank image is used instead of the original blank image in later subtracting steps (i.e., STEP 140).

In some embodiments, the pre-defined block size and the pre-defined block search size are adjustable. In some embodiments, the pre-defined block size used in the block-matching transform is within a range of approximately 1 to approximately 10 pixels. In other words, the block-matching transform includes a block size within a range of approximately 1 to 10 pixels. In some embodiments, the pre-defined block search size used in the block matching transform is within a range of approximately 1 to approximately 10 pixels. In other words, the block-matching transform includes a block search size within a range of approximately 1 to 10 pixels.

In some embodiments, the method for enhancing detection of a target includes any combination of the steps described herein, in various orders. In some embodiments, steps may be omitted. Further, the order of the steps may be reversed, altered, or performed simultaneously.

In at least one embodiment, the electronic-based aspects of the method 100 may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments.

### 6. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1. p-Benzoquinone Treatment in RNAscope® Assays

RNAscope® HiPlex assays were conducted to detect four positive control genes in mouse FFPE kidney tissue. ZZ probes for 12 different targets were hybridized together to a mouse multi-tissue array prepared as formalin-fixed paraffin embedded sections, and the hybridization signals were amplified together by successive rounds of hybridization with the preamplifier and amplifier sequences. Autofluorescence quenching was performed by application of p-benzoquinone (100 mM in 4× Saline-Sodium Citrate (SSC)) onto the slides for 30 minutes at room temperature, followed by detection of four (Ppib, Ubc, Polr2a and Hprt1) of the 12 target genes using fluorescent label probes corresponding to the signal amplification systems assigned to these four target probes and the fluorophores Alexa 488, DyLight 550, DyLight 650 and Alexa 750 respectively (bottom panels). On a separate slide, 4×SSC buffer alone was used as the untreated control. Nuclei were stained with DAPI. As shown in FIG. 3, the p-benzoquinone treatment effectively reduced autofluorescence in the mouse FFPE tissues.

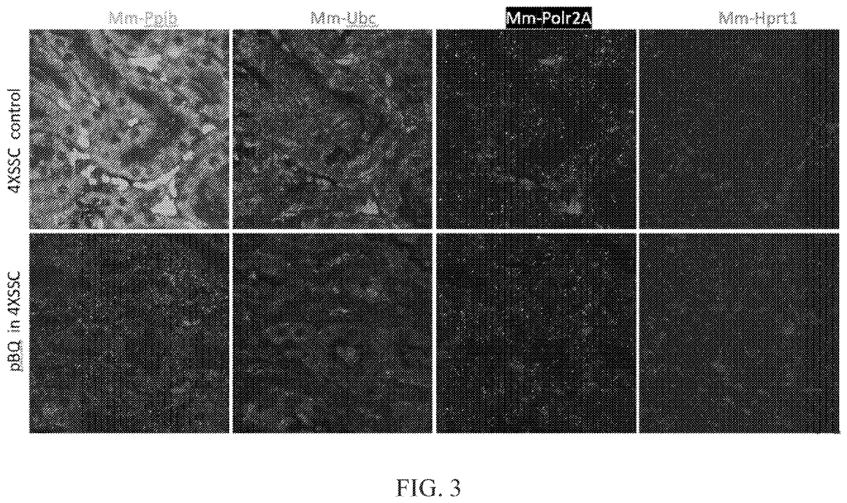

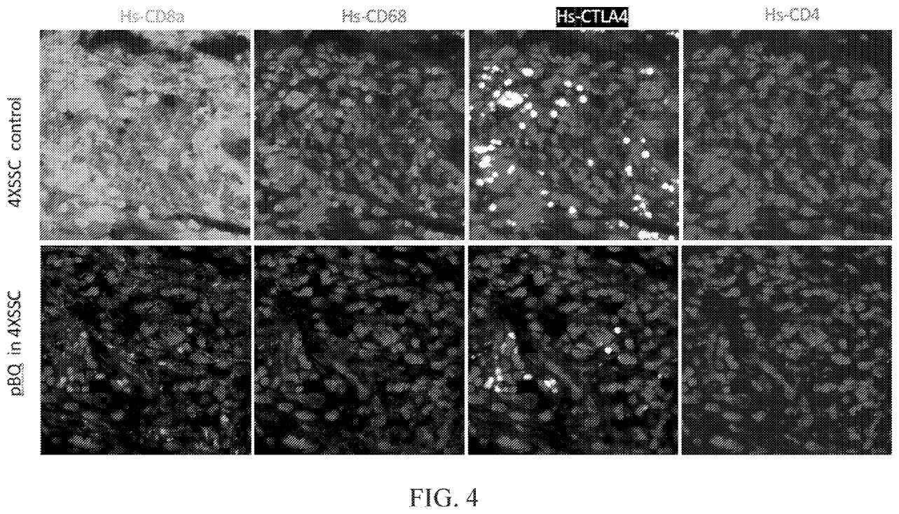

A similar experiment was performed using human FFPE lung cancer tissue, with detection of four immune-oncology markers. Shown in FIG. 4 are representative images for the staining results of four of the twelve genes detected in the third round of HiPlex detection (CD8A, CD68, CTLA4 and CD4), for samples treated with either p-benzoquinone (100 mM in 4×SSC), or control solution (4×SSC alone).

Another similar experiment was performed using non-human primate fixed frozen liver tissue, with detection of four of the twelve targets (POLR2A, PPIB, UBC, and HPRT1). Shown in FIG. 5 are representative images for the staining of these targets, for samples treated with either p-benzoquinone (100 mM in 4×SSC), or control solution (4×SSC alone).

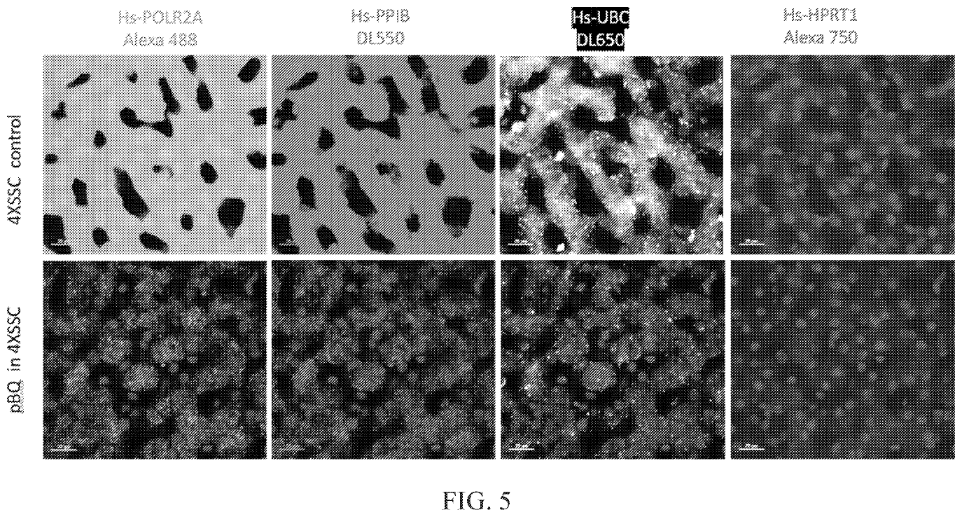

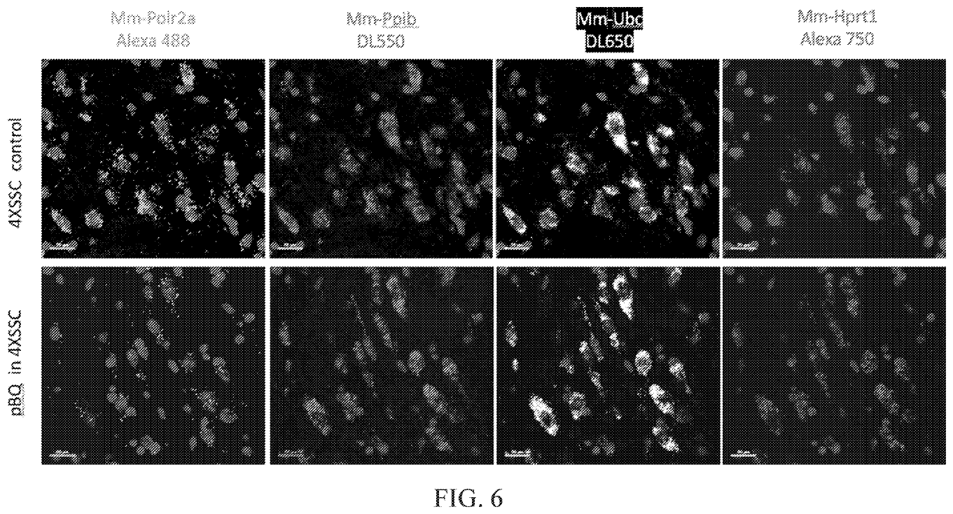

Yet another similar experiment was conducted using mouse fresh frozen tissue, with detection of four of the twelve targets (Polr2a, Ppib, Ubc, and Hprt1). FIG. 6 shows representative images for the staining of these targets, for samples treated with either p-benzoquinone (100 mM in 4×SSC), or control solution (4×SSC alone).

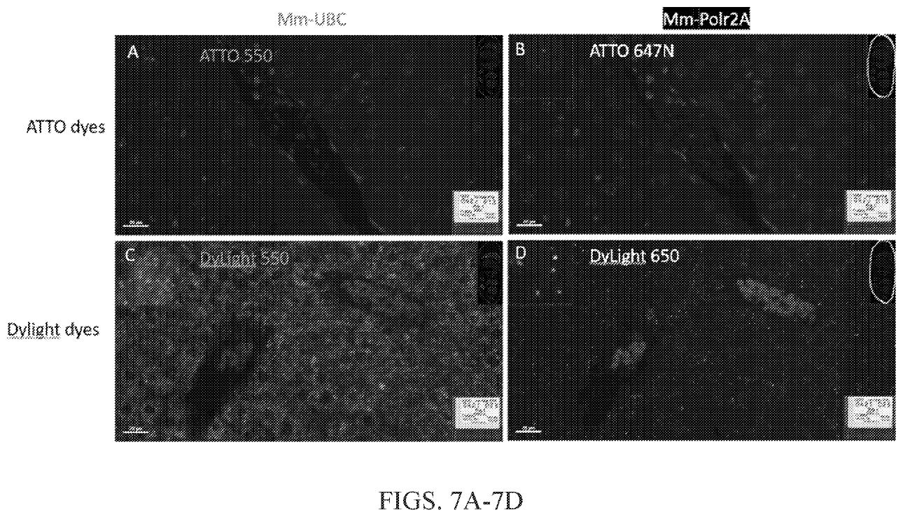

To evaluate the compatibility of certain dyes, targets were detected in mouse FFPE liver tissue, using the p-benzoquinone treatment step. FIG. 7A shows detection of UBC with an ATTO 550 label probe, and FIG. 7B shows detection of POLR2A with an ATTO 647N label probe. FIG. 7C shows detection of UBC with a Dylight 550 label probe, and FIG. 7D shows detection of POLR2A with a Dylight 650 label probe. Experimentally, these targets were detected using RNAscope® double Z probes and signals were amplified using an RNAscope® amplification system (Amp 1, 2, 3), with p-benzoquinone applied after Amp 3. The ATTO dyes did not lead to successful detection of the targets in the images, but signals from the Dylight probes were successfully detected.

### Example 2. p-Benzoquinone Treatment in Immunofluorescence Assays

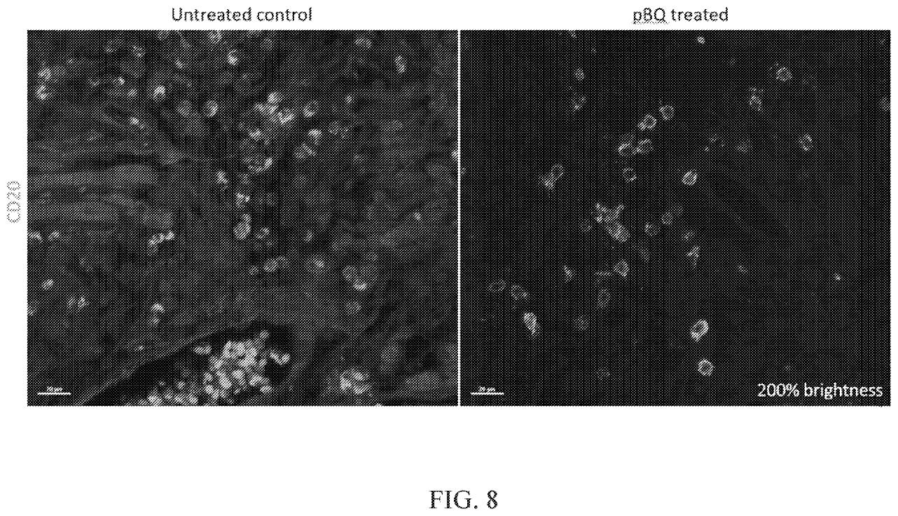

CD20 was detected in in human head and neck FFPE tissue. After bake, deparaffmization, and pretreatment to permeabilize the tissue, 4×SSC (untreated control) or p-benzoquinone (pBQ) (40 mM in 4×SSC) was applied onto the tissue and incubated for 30 min. After washing, mouse anti-CD20 primary antibody (Leica Ready to Use) was applied for 1 hour at room temperature (RT). Signal was amplified by rabbit anti-mouse post primary antibody (Leica Ready to Use) and goat anti-rabbit Alexa 488 secondary antibody (Leica Ready to Use) conjugated to HRP. Signal was detected using Opal 520 (Akoya Bio) diluted 1:500 in TSA buffer. Images are shown in FIG. 8. The images were taken with the same exposure settings. The brightness of the pBQ treated slide was increased by two-fold to achieve optimal display. The data show that p-benzoquinone autofluorescence quenching treatment is compatible with immunofluorescence assays.

Additional antibodies were tested for compatibility with p-benzoquinone treatment, using the same general experimental method as described above. Results are shown in Table 1.

TABLE 1
Effect of pBQ on various antibodies

| | | | | IF Signal |
|---|---|---|---|---|
| Antibody | Tissue | | 2° Ab | compared |
| Tested | Type | 1° Ab | 1:500 | to control |
| NeuN | FFPE mouse | Chicken anti | Goat anti- | Weaker |
| | brain | NeuN 1:500 | chicken AF555 | |
| GFAP | FFPE mouse | Rabbit anti | Goat anti- | Slightly |
| | brain | GFAP 1:1000 | rabbit AF647 | Weaker |
| Ki67 | hTMAc | Rabbit anti | Goat anti- | Incom- |
| | | Ki67 1:500 | rabbit AF647 | patible |
| CD3 | hTMAc | Leica mouse | Goat anti- | Weaker |
| | | anti-CD3 1:200 | rabbit AF488 | |
| CD20 | hTMAc | Leica mouse | Goat anti- | Compa- |
| | | anti-CD20 1:200 | rabbit AF488 | rable |

### Example 3. Quenching of Lipofuscin Autofluorescence

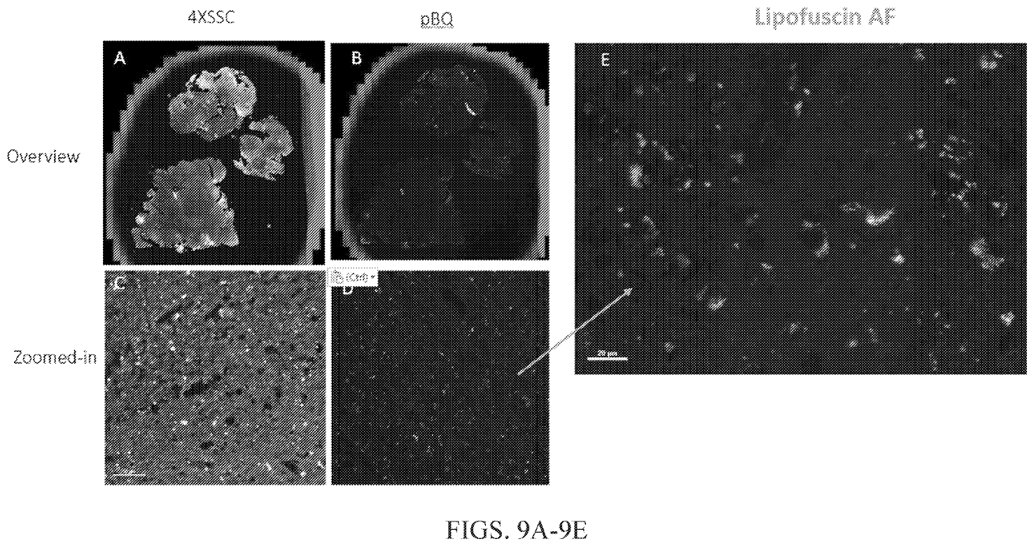

The present methods are effective at partially reducing autofluorescence caused by lipofuscin pigment granules that are most commonly found in brain tissues. FFPE Human Brain Tissues were treated with or without pBQ and imaged without staining to reveal tissue endogenous autofluorescence. Images are shown in FIGS. 9A-9E. p-Benzoquinone at maximum solubility (100 mM in 4×SSC) greatly reduced fixation induced haze background (B and D) compared to no pBQ control (A and C). The dot-like and clusters of fluorcscence resembling lipofuscin was reduced but not completely eliminated (E).

### Example 4. Quinone Compound Testing and Condition Optimization

In a series of experiments to explore optimal quinone treatment conditions, human or mouse formalin fixed paraffin-embedded tissue samples were stained for various positive control genes with the RNAscope HiPlex protocol following the workflow in FIG. 2. Major factors tested for hydroquinone were chemical concentration, incubation time, buffer, light exposure, and workflow position. Major factors tested for p-benzoquinone were chemical concentration, incubation time, and fluorescent dyes. Results are summarized in Table 2. For the signal/noise ratio, −−− indicates no change compared to control, −−+ indicates slight improvement compared to control, −++ indicates improvement compared to control, and +++ indicates the condition was most effective.

TABLE 2
Quinone Treatment Conditions

| | | Target | | | Signal/ |
|---|---|---|---|---|---|
| Chemical | Sample | genes | Factor | Condition | noise ratio |
| Hydroquinone | FFPE mouse | Polr2a, | Chemical | 20 mM, 2 h | −−− |
| | multi tissue | PPIB, UBC, | concentration | 20 mM, 4 d | −−+ |
| | array | Hprt1 | and incubation | 20 mM, 6 d | −−+ |
| | | | time | 200 mM, 2 d | −−+ |
| | | | (with light | 200 mM, 4 d | −++ |
| | | | exposure) | 200 mM, 5 d | −++ |
| | | | | 200 mM, 6 d | −++ |
| | | | Buffer | 2x SSC | −−+ |
| | | | (20 mM, 4 d, with | 4x SSC | −−+ |
| | | | light exposure) | Decal | −−+ |
| | | | | RNAase | −−+ |
| | | | | later/inhibitor | |
| | | | Light exposure | Yes | −−+ |
| | | | (200 mM, 2 d) | No | −−− |
| | | | Workflow | After target | −−− |
| | | | position | probe | |
| | | | (20 mM, 2 d, with | After Amp1 | −−+ |
| | | | light exposure) | After Amp2 | −−+ |
| | | | | After Amp3 | −++ |
| p-benzoquinone | FFPE mouse | Polr2a, | Chemical | 2 mM, overnight | +++ |
| (pBQ) | multi tissue | PPIB, UBC, | concentration | 20 mM, 30 min | −−+ |
| | array | Hprt1 | and incubation | 100 mM, 30 min | +++ |
| | | | time | 200 mM, 30 min | +++ |
| | | | Fluorescent dyes | ATTO dyes | −−− |
| | | | | CY dyes | −−− |
| | | | | Dylight dyes | +++ |
| | | | | Alexa dyes | +++ |
| | FFPE human | Polr2a, | Chemical | 100 mM, 30 min | +++ |
| | tumor | PPIB, UBC, | concentration | | |
| | microarray | Hprt1 | and incubation | | |
| | | | time | | |

Multiple quinone compounds were tested for tissue autofluorescence quenching effects on FFPE mouse multi-tissue arrays. After bake, deparaffinization, and pretreatment to permeabilize the tissue, 4×SSC or quinone derivative was applied to the tissue and incubated for 30 min. After washing DAPI was applied to stain the nucleus. Slides were then mounted with prolong gold and scanned using the same microscopic settings. A reduction in autofluorescence was determined by comparing the fluorescence on the quinone derivative treated slide with a 4×SSC untreated control slide. Results are summarized in Table 3.

TABLE 3
Quinone Compounds Tested for Tissue
Autofluorescence Quenching

| | Autofluorescence |
|---|---|
| Chemical name | quenching effect |
| Hydroquinone | Yes |
| p-benzoquinone | Yes |
| Tetrahydroxy-1,4-quinone hydrate | No |
| 2,3-Dichloro-5,8-dihydroxy-1,4-naphthoquinone | Yes, but spectrum |
| | shift to Cy5 channel |
| 2-methoxybenzo-1,4-quinone | Yes |
| 2-Chloro-1,4-benzoquinone | Yes |
| 2,5-Dichloro-1,4-benzoquinone | Yes |
| 2,6-Dichloro-1,4-benzoquinone | Yes |
| 2,6-Dimethoxy-1,4-benzoquinone | No |
| 2,5-Dimethoxy-(1,4)-benzoquinone | No |
| Methyl-p-benzoquinone | No |
| 2,5-Dimethyl-1,4-benzoquinone | Partial |
| 2,6-Dimethylbenzoquinone | No |

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A method of detecting a target in a biological sample, the method comprising:
contacting the sample with a probe that binds to the target;
contacting the sample with a quinone compound; and
detecting a signal corresponding to the target in the sample.

2. The method of claim 1, wherein the target is a nucleic acid target.

3. The method of claim 2, wherein the probe is a first target probe comprising a T section and an L section, wherein the T section is complementary to a portion of the nucleic acid target and wherein the L section is complementary to a nucleic acid component of a signal generating complex.

4. The method of claim 3, further comprising contacting the sample with a second target probe comprising a T section and an L section, wherein the T section is complementary to a portion of the nucleic acid target and wherein the L section is complementary to a nucleic acid component of a signal generating complex.

5. The method of claim 4, wherein the T sections of the first target probe and the second target probe are complementary to non-overlapping regions of the nucleic acid target.

6. The method of claim 4 or claim 5, wherein the L sections of the first target probe and the second target probe are complementary to non-overlapping regions of the signal generating complex.

7. The method any one of claims 3-6, further comprising contacting the sample with a signal generating complex comprising at least one detectable label.

8. The method of claim 7, wherein the detecting step comprises detecting a signal generated by the signal generating complex.

9. The method of claim 7 or claim 8, wherein the signal generating complex comprises a label probe that binds to the detectable label, and optionally, one or more of an amplifier, a pre-amplifier, and a pre-pre-amplifier.

10. The method of claim 9, wherein the signal generating complex comprises a label probe, an amplifier, and a pre-amplifier.

11. The method of any one of claims 7-10, wherein the sample is contacted with the quinone compound after the sample has been contacted with the first target probe and the second target probe, but before the sample is contacted with the detectable label.

12. The method of any one of claims 7-11, wherein the detectable label is a fluorophore or a quantum dot.

13. The method of claim 12, wherein the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

14. The method of any one of claims 2-13, wherein the nucleic acid target is selected from an RNA and a DNA.

15. The method of claim 14, wherein the nucleic acid target is an RNA, and the RNA is selected from messenger RNA (mRNA), microRNA (miRNA), ribosomal RNA (rRNA), mitochondrial RNA, and non-coding RNA.

16. The method of claim 15, wherein the nucleic acid target is mRNA.

17. The method of claim 14, wherein the nucleic acid target is DNA.

18. The method of claim 1, wherein the target is a protein.

19. The method of claim 18, wherein the probe is a primary antibody that binds to the protein target, and the method further comprises contacting the sample with a secondary antibody that binds to the primary antibody, wherein the secondary antibody binds to a detectable label.

20. The method of any one of claims 1-19, wherein the method comprises detecting multiple targets in the biological sample.

21. The method of claim 21, wherein the method comprises detecting multiple nucleic acid targets, multiple protein targets, or at least one each of a nucleic acid target and a protein target.

22. The method of any one of claims 1-21, wherein the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone.

23. The method of claim 22, wherein the quinone compound is p-benzoquinone or hydroquinone.

24. The method of any one of claims 1-23, wherein the sample is a tissue specimen or is derived from a tissue specimen.

25. The method of claim 24, wherein the sample is a formalin fixed paraffin embedded tissue specimen, a fixed frozen tissue specimen, or a fresh frozen tissue specimen.

26. The method of claim 25, wherein the sample is a formalin fixed paraffin embedded tissue specimen, and the method further comprises performing a deparaffmization step prior to the contacting or detecting steps.

27. A kit for detecting a target in a biological sample, the kit comprising:
a probe that binds to the target;
a quinone compound; and
instructions for conducting an assay to detect the target in the biological sample.

28. The kit of claim 27, wherein the target is a nucleic acid target.

29. The kit of claim 28, wherein the probe is a first target probe comprising a T section and an L section, wherein the T section is complementary to a portion of the nucleic acid target and wherein the L section is complementary to a nucleic acid component of a signal generating complex.

30. The kit of claim 29, further comprising a second target probe comprising a T section and an L section, wherein the T section is complementary to a portion of the nucleic acid target and wherein the L section is complementary to a nucleic acid component of a signal generating complex.

31. The kit of claim 30, wherein the T sections of the first target probe and the second target probe are complementary to non-overlapping regions of the nucleic acid target.

32. The kit of claim 30 or claim 31, wherein the L sections of the first target probe and the second target probe are complementary to non-overlapping regions of the signal generating complex.

33. The kit any one of claims 29-32, further comprising a signal generating complex comprising at least one detectable label.

34. The kit of claim 33, wherein the signal generating complex comprises a label probe that binds to the detectable label, and optionally, one or more of an amplifier, a pre-amplifier, and a pre-pre-amplifier.

35. The kit of claim 34, wherein the signal generating complex comprises a label probe, an amplifier, and a pre-amplifier.

36. The kit of any one of claims 33-35, wherein the detectable label is a fluorophore or a quantum dot.

37. The kit of claim 36, wherein the detectable label is a fluorophore selected from a fluorescein, a rhodamine, a boron-dipyrromethene, a cyanine, an oxazine, a thiazine, a coumarin, a naphthalimide, a rhodol, a naphthalene, a squaraine, a porphyrin, and a flavin.

38. The kit of any one of claims 28-37, wherein the nucleic acid target is selected from an RNA and a DNA.

39. The kit of claim 38, wherein the nucleic acid target is an RNA, and the RNA is selected from messenger RNA (mRNA), microRNA (miRNA), ribosomal RNA (rRNA), mitochondrial RNA, and non-coding RNA.

40. The kit of claim 39, wherein the nucleic acid target is mRNA.

41. The kit of claim 38, wherein the nucleic acid target is DNA.

42. The kit of claim 27, wherein the target is a protein.

43. The kit of claim 42, wherein the probe is a primary antibody that binds to the protein target, and the kit further comprises a secondary antibody that binds to the primary antibody, wherein the secondary antibody binds to a detectable label.

44. The kit of any one of claims 27-43, further comprising a second probe for a second target in the biological sample.

45. The kit of claim 44, wherein the second target is a nucleic acid or a protein.

46. The kit of any one of claims 27-45, wherein the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone.

47. The kit of claim 46, wherein the quinone compound is p-benzoquinone or hydroquinone.

48. The kit of any of claims 27-47, wherein the kit further comprises at least one of a hybridization buffer, dextran sulfate, formamide, dithiothreitol (DDT), sodium chloride and sodium citrate (SSC), EDTA, Denhardt's solution, dNTPs, single-stranded DNA, tRNA, polyA, an initiator oligo, or any combination thereof.

49. A composition comprising:
a biological sample comprising a target;
a probe that binds to the target; and
a quinone compound.

50. The composition of claim 49, wherein the target is a nucleic acid target.

51. The composition of claim 50, wherein the probe is a first target probe comprising a T section and an L section, wherein the T section is complementary to a portion of the nucleic acid target and wherein the L section is complementary to a nucleic acid component of a signal generating complex.

52. The composition of claim 51, further comprising a second target probe comprising a T section and an L section, wherein the T section is complementary to a portion of the nucleic acid target and wherein the L section is complementary to a nucleic acid component of a signal generating complex.

53. The composition of claim 52, wherein the T sections of the first target probe and the second target probe are complementary to non-overlapping regions of the nucleic acid target.

54. The composition of claim 52 or claim 53, wherein the L sections of the first target probe and the second target probe are complementary to non-overlapping regions of the signal generating complex.

55. The composition any one of claims 51-54, further comprising a signal generating complex comprising at least one detectable label.

56. The composition of claim 55, wherein the signal generating complex comprises a label probe that binds to the detectable label, and optionally, one or more of an amplifier, a pre-amplifier, and a pre-pre-amplifier.

57. The composition of claim 56, wherein the signal generating complex comprises a label probe, an amplifier, and a pre-amplifier.

58. The composition of any one of claims 55-57, wherein the detectable label is a fluorophore or a quantum dot.

59. The composition of claim 58, wherein the detectable label is a fluorophore selected from a fluorescein, a rhodamine, a boron-dipyrromethene, a cyanine, an oxazine, a thiazine, a coumarin, a naphthalimide, a diodol, a naphthalene, a squaraine, a porphyrin, and a flavin.

60. The composition of any one of claims 49-59, wherein the nucleic acid target is selected from an RNA and a DNA.

61. The composition of claim 60, wherein the nucleic acid target is an RNA, and the RNA is selected from messenger RNA (mRNA), microRNA (miRNA), ribosomal RNA (rRNA), mitochondrial RNA, and non-coding RNA.

62. The composition of claim 61, wherein the nucleic acid target is mRNA.

63. The composition of claim 60, wherein the nucleic acid target is DNA.

64. The composition of claim 49, wherein the target is a protein.

65. The composition of claim 64, wherein the probe is a primary antibody that binds to the protein target, and the composition further comprises a secondary antibody that binds to the primary antibody, wherein the secondary antibody binds to a detectable label.

66. The composition of any one of claims 49-65, wherein the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone.

67. The composition of claim 66, wherein the quinone compound is p-benzoquinone or hydroquinone.

68. The composition of any one of claims 49-67, wherein the biological sample is a tissue specimen or is derived from a tissue specimen.

69. The composition of claim 68, wherein the sample is a formalin fixed paraffin embedded tissue specimen, a fixed frozen tissue specimen, or a fresh frozen tissue specimen.

70. The composition of any one of claims 49-67, wherein the biological sample is a cytological sample or is derived from a cytological sample.

71. A method of reducing autofluorescence in a biological sample, comprising contacting the sample with an effective amount of a quinone compound.

72. The method of claim 71, wherein the quinone compound is selected from the group consisting of p-benzoquinone, hydroquinone, 2,3-dichloro-5,8-dihydroxy-1,4-napthoquinone, 2-methoxybenzo-1,4-quinone, 2-chloro-1,4-benzoquinone, 2,5-dichloro-1,4-benzoquinone, 2,6-dichloro-1,4-benzoquinone, and 2,5-dimethyl-1,4-benzoquinone.

73. The method of claim 71 or claim 72, wherein the quinone compound is p-benzoquinone or hydroquinone.

74. The method of any one of claims 71-73, wherein the sample is a tissue specimen or is derived from a tissue specimen.

75. The method of claim 74, wherein the sample is a formalin fixed paraffin embedded tissue specimen, a fixed frozen tissue specimen, or a fresh frozen tissue specimen.
